(19) United States
(12) Patent Application Publication
(10) Pub. No.: US 2023/0333126 A1
(43) Pub. Date: Oct. 19, 2023

(54) MARKERS FOR DETECTING CARDIOVASCULAR DISEASE AND CARDIOVASCULAR DISEASE RISK

(71) Applicant: The Regents of the University of Michigan, Ann Arbor, MI (US)

(72) Inventor: Michael Holinstat, Ann Arbor, MI (US)

(21) Appl. No.: 18/027,764

(22) PCT Filed: Sep. 24, 2021
(86) PCT No.: PCT/US2021/051983

§ 371 Date: Mar. 22, 2023

Related U.S. Application Data
(60) Provisional application No. 63/083,535, filed on Sep. 25, 2020.

(51) Int. Cl. A61K 45/06; G01N 33/92

(52) U.S. Cl. CPC G01N 33/92; A61K 45/06; G01N 2800/32; G01N 2800/52

(57) ABSTRACT

Provided herein are compositions, systems, kits, and methods for detecting cardiovascular disease, and risk of cardiovascular disease in a subject based on the levels of 12-(S)-hydroxyeicosatetraenoic acid (12(S)-HETE) and/or 3-Hydroxyoctadecadienoic acid (13-HODE) in the subject.

### CROSS REFERENCE TO RELATED APPLICATION

This application claims priority to U.S. Provisional Pat. Application Serial No. 63/083,535 filed Sep. 25, 2020, which is incorporated herein by reference in its entirety.

### FEDERALLY SPONSORED RESEARCH OR DEVELOPMENT

This invention was made with government support under GM131835 awarded by the National Institutes of Health. The government has certain rights in this invention.

### FIELD OF THE INVENTION

Provided herein are compositions, systems, kits, and methods for detecting cardiovascular disease, and risk of cardiovascular disease in a subject based on the levels of 12-(S)-hydroxyeicosatetraenoic acid (12(S)-HETE) and/or 3-Hydroxyoctadecadienoic acid (13-HODE) in the subject.

### INTRODUCTION

Improved methods and techniques for detecting, treating and preventing cardiovascular thrombosis are needed.

The present invention addresses such needs.

### SUMMARY

Experiments conducted during the course of developing embodiments for the present invention identification of 12(S)-HETE and/or 13-HOTE as a marker for detecting the presence of and risk of developing cardiovascular thrombosis. In particular, such experiments involved a longitudinal testing of patients diagnosed with COVID-19 at high risk for blood coagulation issues. It was shown that 12(S)-HETE and 13-HOTE levels are elevated in patients at a high risk for cardiovascular issues and thrombosis. It was further shown that high 12(S)-HETE levels are predictive of platelet activation in the blood since they are the only cell in the blood that can produce 12(S)-HETE.

Accordingly, provided herein are compositions, systems, kits, and methods for detecting cardiovascular disease, and risk of cardiovascular disease in a subject based on the levels of 12-(S)-hydroxyeicosatetraenoic acid (12(S)-HETE) and/or 3-Hydroxyoctadecadienoic acid (13-HODE) in the subject.

### BRIEF DESCRIPTION OF DRAWINGS

FIG. 1 shows in patients at high risk for blood coagulation issues through longitudinal testing of patients in the hospital with COVID-19 that 12(S)-HETE levels are elevated in these patients that are at a high risk for cardiovascular issues and thrombosis and that the elevated levels are measured upon first blood sample submitted to the coagulation lab at the hospital for PTT testing.

FIG. 2: 12-HETE and 13-HODE levels were measured by LC-MS/MS from the plasma of healthy subjects and COVID-19 patients in the ICU. The level of 12-HETE and 13-HODE in the COVID-19 patients was significantly higher than that from healthy subjects suggesting highly active platelets and immune cells.

FIG. 3: The levels of a number of eicosanoids were measured in the plasma from COVID-19 patients throughout their treatment in the ICU at the University of Michigan Hospital. While TxB2 levels remained relatively low, 12-HETE and 13-HODE levels significantly increased over time. 14-HDHA levels also showed an increase to a lower degree.

FIG. 4: Oxygen levels were observed to decrease several days following the increase in 12-HETE levels suggesting a 12-HETE could be a diagnostic indicator for thrombotic risk with oxygen availability decreasing once the thrombosis presents

### DETAILED DESCRIPTION

12-lipoxygenase (12-LOX) is an enzyme primarily located and expressed in the human platelet. 12-LOX is a constitutively active enzyme that adds an oxygen to the 12^{th} carbon of arachidonic acid when it is freed from the plasma membrane following initial platelet activation in the blood. When 12-LOX adds an oxygen to the 12^{th} carbon of arachidonic acid, the product is called 12-(S)-hydroxyeicosatetraenoic acid or 12(S)-HETE. 12(S)-HETE ranges from the pM levels in unstimulated blood to the uM levels in fully activated platelets in blood. It is known that 12(S)-HETE is very stable in blood and can be measured reproducibly in urine as well.

Experiments conducted during the course of developing embodiments for the present invention identification of 12(S)-HETE and/or 13-HODE as a marker for detecting the presence of and risk of developing cardiovascular thrombosis. In particular, such experiments involved a longitudinal testing of patients diagnosed with COVID-19 at high risk for blood coagulation issues. It was shown that 12(S)-HETE and 13-HODE levels are elevated in patients at a high risk for cardiovascular issues and thrombosis. It was further shown that high 12(S)-HETE levels are predictive of platelet activation in the blood since they are the only cell in the blood that can produce 12(S)-HETE.

Accordingly, provided herein are compositions, systems, kits, and methods for detecting cardiovascular disease, and risk of cardiovascular disease in a subject based on the levels of 12-(S)-hydroxyeicosatetraenoic acid (12(S)-HETE) and/or 3-Hydroxyoctadecadienoic acid (13-HODE) in the subject.

In some embodiments, provided herein are methods for performing an activity based on concentration level of 12(S)-HETE and/or 13-HODE in a biological sample from a subject comprising: a) determining the concentration level of total 12(S)-HETE and/or 13-HODE in a biological sample from a subject; and b) performing at least one of the following: i) identifying increased 12(S)-HETE and/or 13-HODE expression level total (e.g., compared to control 12(S)-HETE and/or 13-HODE expression levels from disease free or general population; ii) generating and/or transmitting a report that indicates the 12(S)-HETE and/or 13-HODE expression levels are increased (e.g., compared to control 12(S)-HETE and/or 13-HODE expression levels from disease free or general population) in the sample, and that the subject is in need of a CVD therapeutic agent; and iii) generating and/or transmitting a report that indicates the total 12(S)-HETE and/or 13-HODE expression levels are increased (e.g., compared to control 12(S)-HETE and/or 13-HODE expressoin levels from disease free or general population) in the sample, and that the subject has or is at risk of cardiovascular disease (e.g., cardiovascular thrombosis) (e.g., any cardiovascular disorder characterized with increased 12(S)-HETE and/or 13-HODE activity) or complication of cardiovascular disease.

In certain embodiments, the CVD therapeutic agent is selected from the group consisting of: an antibiotic, a statin, a probiotic, an alpha-adrenergic blocking drug, an angiotensin-converting enzyme inhibitor, an angiotensin receptor antagonist, an antiarrhythmic drug, an anticoagulant, an antiplatelet drug, a thromybolytic drug, a beta-adrenergic blocking drug, a calcium channel blocker, a brain acting drug, a cholesterol-lowering drug, a TMEM55b inhibitor, a OCRL1 inhibitor, a digitalis drug, a diuretic, a nitrate, a peripheral adrenergic antagonist, and a vasodilator. In particular embodiments, the subject is a human. In other embodiments, the biological sample is a plasma, serum, blood, urine, or similar sample.

In certain embodiments, the cardiovascular disease or complication of cardiovascular disease is cardiovascular thrombosis or any cardiovascular disorder characterized with increased 12(S)-HETE and/or 13-HODE activity levels.

In some embodiments, provided herein are systems comprising: a) a report for a subject indicating that the subject has increased 12(S)-HETE and/or 13-HODE activity levels; and b) a CVD therapeutic agent.

In certain embodiments, provided herein are methods comprising: a) identifying a subject as having increased 12(S)-HETE and/or 13-HODE expression levels, and b) treating the subject with a CVD therapeutic agent. In further embodiments, the identifying comprises receiving the report.

In some embodiments, provided herein are methods for evaluating the effect of a cardiovascular disease (CVD) therapeutic agent on a subject comprising: a) determining a first 12(S)-HETE and/or 13-HODE expression level in a bodily sample (e.g., plasma) taken from a subject (e.g., human subject) prior to administration of a CVD therapeutic agent, and b) determining a second 12(S)-HETE and/or 13-HODE expression level in a corresponding bodily fluid taken from the subject following administration of the CVD therapeutic agent.

In certain embodiments, a decrease in the second 12(S)-HETE and/or 13-HODE expression level to the first level is indicative of a positive effect of the CVD therapeutic agent on cardiovascular disease in the subject. In further embodiments, the CVD therapeutic agent comprises a lipid reducing agent (e.g., a statin). In further embodiments, the CVD therapeutic agent is selected from the group consisting of: an anti-inflammatory agent, a TMEM55b inhibitor, a OCRL1 inhibitor, an insulin sensitizing agent, an anti-hypertensive agent, an anti-thrombotic agent, an anti-platelet agent, a fibrinolytic agent, a direct thrombin inhibitor, an ACAT inhibitor, a CETP inhibitor, and a glycoprotein IIb/IIIa receptor inhibitor. In particular embodiments, the CVD is atherosclerotic CVD. In other embodiments, the subject has been diagnosed as having CVD or cardiovascular thrombosis. In further embodiments, the subject has been diagnosed as being at risk of developing CVD or cardiovascular thrombosis. In certain embodiments, the bodily sample is a plasma, blood, serum, urine, or other sample.

In certain embodiments, the present invention provides methods for characterizing a sample obtained from a subject having or at risk of having a cardiovascular thrombosis, comprising measuring a 12(S)-HETE and/or 13-HODE expression level in a sample (e.g., blood, plasma, urine, etc.) from the subject, and determining that the subject is experiencing cardiovascular thrombosis or is at risk of experiencing cardiovascular thrombosis if the measured 12(S)-HETE and/or 13-HODE expression level is higher compared to control 12(S)-HETE and/or 13-HODE activity levels from disease free or general population. In some embodiments, the method further comprises administering a treatment (e.g., a CVD therapeutic agent) to a subject characterized experiencing cardiovascular thrombosis or is at risk of experiencing cardiovascular thrombosis.

In certain embodiments, the present invention provides methods for detecting a cardiovascular thrombosis or risk of developing a cardiovascular thrombosis in a subject having or at risk of having a cardiovascular thrombosis, comprising measuring a 12(S)-HETE and/or 13-HODE expression level in a sample (e.g., blood, plasma, urine, etc.) from the subject, and determining that the subject is experiencing cardiovascular thrombosis or is at risk of experiencing cardiovascular thrombosis if the measured 12(S)-HETE and/or 13-HODE expression level is higher compared to control 12(S)-HETE and/or 13-HODE expression levels from disease free or general population. In some embodiments, the method further comprises administering a treatment (e.g., a CVD therapeutic agent) to a subject characterized experiencing cardiovascular thrombosis or is at risk of experiencing cardiovascular thrombosis.

In certain embodiments, the present invention provides methods for treating and/or preventing a cardiovascular thrombotic state in a subject (e.g., a human subject), comprising administering to a subject experiencing or at risk of experiencing a cardiovascular thrombotic state a therapeutic agent capable of decreasing 12(S)-HETE and/or 13-HODE expression levels and/or inhibiting 12-lipoxygenase activity and/or inhibiting interaction between 12-lipoxygenase and arachidonic acid. Such embodiments are not limited to a particular type of therapeutic agent capable of decreasing 12(S)-HETE and/or 13-HODE expression levels and/or inhibiting 12-lipoxygenase activity and/or inhibiting interaction between 12-lipoxygenase and arachidonic acid (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods for hindering and/or inhibiting blood platelet activation in a subject, comprising administering to a subject experiencing or at risk of experiencing a cardiovascular thrombotic state a therapeutic agent capable of decreasing 12(S)-HETE and/or 13-HODE activity levels and/or inhibiting 12-lipoxygenase activity and/or inhibiting interaction between 12-lipoxygenase and arachidonic acid. Such embodiments are not limited to a particular type of therapeutic agent capable of decreasing 12(S)-HETE and/or 13-HODE activity levels and/or inhibiting 12-lipoxygenase activity and/or inhibiting interaction between 12-lipoxygenase and arachidonic acid (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

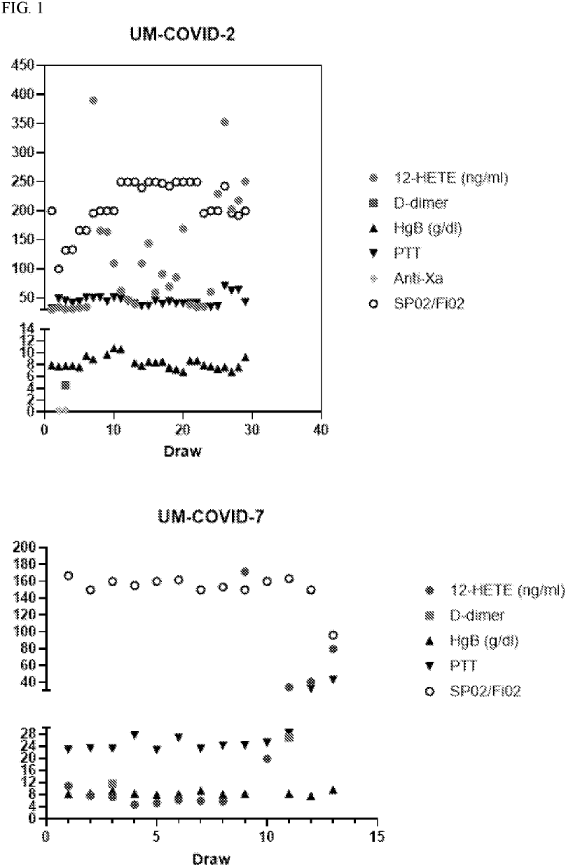

We now show in patients at high risk for blood coagulation issues through longitudinal testing of patients in the hospital with COVID-19 that 12(S)-HETE levels are elevated in these patients that are at a high risk for cardiovascular issues and thrombosis and that the elevated levels are measured upon first blood sample submitted to the coagulation lab at the hospital for PTT testing (see FIG. 1). The level tracks well and is highly sensitive. High 12(S)-HETE levels are predictive of platelet activation in the blood since they are the only cell in the blood that can produce 12(S)-HETE.

The method used to measure 12(S)-HETE in my lab was LC/MS/MS (liquid chromatography tandem Mass Spec) as follows: Extraction of 12S-HETE from COVID plasma.

50 µL aliquots of plasma were briefly spun down in a bench-top centrifuge then added to 2 mL of dichloromethane in 7 mL scintillation vials. 50 ng of 12(S)-HETE-d₈ and 50 ng of Thromboxane B2-d₄ were added to the vials to act as extraction standards. Following the addition of 2 mL of nanopure H₂O, 50 µL of 1 M HCl was added to acidify the solution. The vials were vortexed for 10 seconds and briefly centrifuged. The organic layer containing desired analytes was transferred to a cold vial. The aqueous layer was washed an additional two times with 2 mL of DCM. The samples were then reduced through the addition of 3 drops of trimethyl phosphite and were blown down under N₂ to dryness. In order to precipitate the remaining protein, the samples were reconstituted in 500 uL of ACN:H₂O (4:1), stored at -70C for 20 minutes, thawed, centrifuged at 13000 RPM for 15 minutes at 4C, and transferred to a new vial. The samples were blown down under N₂ to dryness, reconstituted in 50 µL of acetonitrile and 50 µL water with 0.1% formic acid and 90 µL was injected for LC-MS/MS analysis. Structural characterization was performed on a Sciex Excion LC, using a C₁₈ column (Phenomenex Kinetex, 4 µm, 150 mm × 2.0 mm). Mobile phase A consisted of water with 0.1% (v/v) formic acid, and mobile phase B was acetonitrile with 0.1% formic acid. The flow rate was 0.400 mL/min with initial conditions (15% B) maintained for 3 min. Mobile phase B was ramped to 60% over 7.5 min, to 70% over 4.5 min, to 80% over 1 min, to 100% over 1 min, held at 100% for 2 min, and finally returned to 15% to equilibrate for 2 min. The chromatography system was coupled to a Sciex PDA and x500B qTOF. Analytes were ionized through electrospray ionization with a -4.0 kV spray voltage and 50, 50, and 20 PSI for ion source gas 1, 2, and curtain gas respectively. The CAD gas was set to 7 while the probe temperature was 550° C., respectively. DP was -60 V and CE was set to -10 V with a 5 V spread. MS² acquisition was performed using SWATH and m/z ratios ± 0.5: 295.228 (HODE’s), 319.228 (HETE’s), 327.290 (12(S)-HETE-d₈), 343.228 (HDHA’s), 345.224 (DPAₙ₋₃), 369.228 (TXB2), and 373.260 (TXB2-d₄) were used. All analyses were performed in negative ionization mode at the normal resolution setting. Matching retention times and fragmentation patterns to known standards with at least 6 common fragments identified the products.

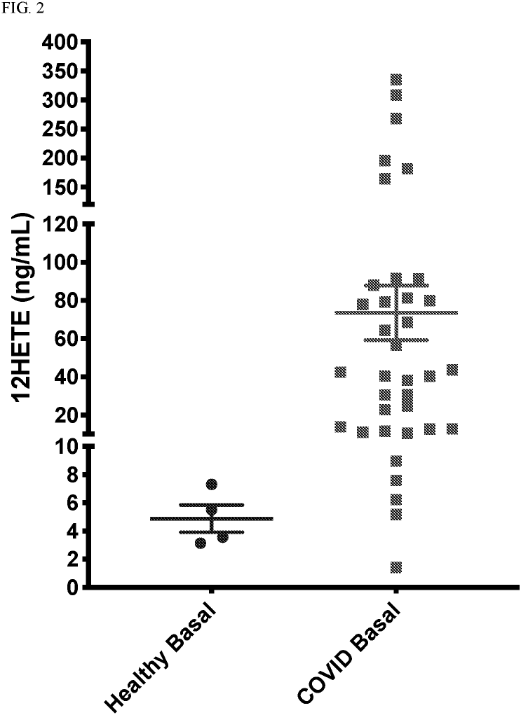

Additional experiments were conducted wherein 12-HETE and 13-HODE levels were measured by LC-MS/MS from the plasma of healthy subjects and COVID-19 patients in the ICU. As shown in FIG. 2, the level of 12-HETE and 13-HODE in the COVID-19 patients was significantly higher than that from healthy subjects suggesting highly active platelets and immune cells.

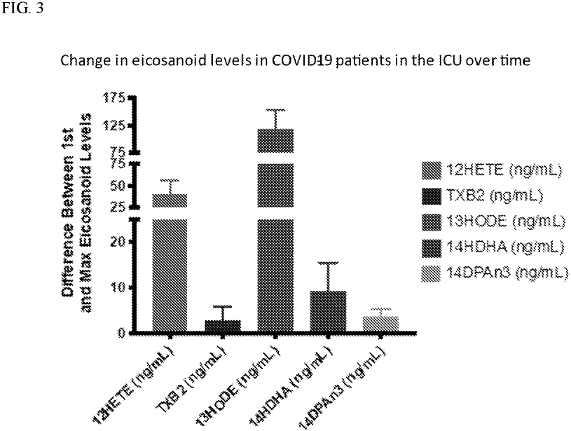

Additional experiments were conducted wherein the levels of a number of eicosanoids were measured in the plasma from COVID-19 patients throughout their treatment in the ICU at the University of Michigan Hospital. As shown in FIG. 3, while TxB2 levels remained relatively low, 12-HETE and 13-HODE levels significantly increased over time. 14-HDHA levels also showed an increase to a lower degree.

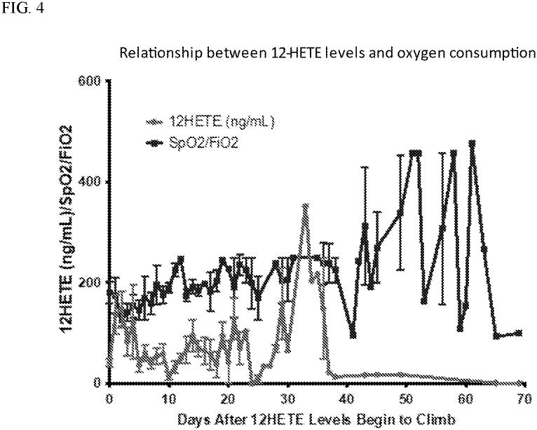

Additional experiments were conducted wherein oxygen levels were observed to decrease several days following the increase in 12-HETE levels indicating 12-HETE as a diagnostic indicator for thrombotic risk with oxygen availability decreasing once the thrombosis presents (FIG. 4).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method for performing an activity based on concentration level of 12(S)-HETE and/or 13-HODE in a biological sample (e.g., plasma sample, blood platelet sample, urine sample, etc.) from a subject comprising:
a) determining the concentration level of total 12(S)-HETE and/or 13-HODE in a biological sample from a subject; and
b) performing at least one of the following:
i) identifying increased 12(S)-HETE and/or 13-HODE expression level total (e.g., compared to control 12(S)-HETE and/or 13-HODE expression levels from disease free or general population), and treating the subject with a CVD (cardiovascular disease) therapeutic agent;
ii) generating and/or transmitting a report that indicates the 12(S)-HETE and/or 13-HODE expression levels are increased (e.g., compared to control 12(S)-HETE and/or 13-HODE activity levels from disease free or general population) in the sample, and that the subject is in need of a CVD therapeutic agent; and
iii) generating and/or transmitting a report that indicates the total 12(S)-HETE expression levels are increased (e.g., compared to control 12(S)-HETE and/or 13-HODE expression levels from disease free or general population) in the sample, and that the subject has or is at risk of cardiovascular disease (e.g., cardiovascular thrombosis) (e.g., any cardiovascular disorder characterized with increased 12(S)-HETE and/or 13-HODE expression) or complication of cardiovascular disease.

2. The method of claim 1, wherein the CVD therapeutic agent is selected from the group consisting of: an antibiotic, a statin, a probiotic, an alpha-adrenergic blocking drug, an angiotensin-converting enzyme inhibitor, an angiotensin receptor antagonist, an antiarrhythmic drug, an anticoagulant, an antiplatelet drug, a thromybolytic drug, a beta-adrenergic blocking drug, a calcium channel blocker, a brain acting drug, a cholesterol-lowering drug, a TMEM55b inhibitor, a OCRL1 inhibitor, a digitalis drug, a diuretic, a nitrate, a peripheral adrenergic antagonist, and a vasodilator.

3. The method of claim 1, wherein the subject is a human.

4. The method of claim 1, wherein the biological sample is a plasma, serum, blood, urine, or similar sample.

5. The method of claim 1, wherein the cardiovascular disease or complication of cardiovascular disease is cardiovascular thrombosis or any cardiovascular disorder characterized with increased 12(S)-HETE expression levels.

6. A system comprising: a) a report for a subject indicating that the subject has increased 12(S)-HETE and/or 13-HODE activity levels; and b) a CVD therapeutic agent.

7. A method comprising:
a) identifying a subject as having increased 12(S)-HETE and/or 13-HODE expression levels, and
b) treating the subject with a CVD therapeutic agent.

8. A method for evaluating the effect of a cardiovascular disease (CVD) therapeutic agent on a subject comprising:
a) determining a first 12(S)-HETE and/or 13-HODE expression level in a bodily sample (e.g., plasma) taken from a subject (e.g., human subject) prior to administration of a CVD therapeutic agent, and
b) determining a second 12(S)-HETE and/or 13-HODE expression level in a corresponding bodily fluid taken from the subject following administration of the CVD therapeutic agent;
wherein a decrease in the second 12(S)-HETE and/or 13-HODE expression level to the first level is indicative of a positive effect of the CVD therapeutic agent on cardiovascular disease in the subject.

9. The method of claim 8, wherein the CVD therapeutic agent is selected from the group consisting of a lipid reducing agent (e.g., a statin), an anti-inflammatory agent, a TMEM55b inhibitor, a OCRL1 inhibitor, an insulin sensitizing agent, an anti-hypertensive agent, an antithrombotic agent, an anti-platelet agent, a fibrinolytic agent, a direct thrombin inhibitor, an ACAT inhibitor, a CETP inhibitor, and a glycoprotein IIb/IIIa receptor inhibitor.

10. The method of claim 9, wherein the subject has been diagnosed as having CVD or cardiovascular thrombosis.

11. The method of claim 9, wherein the subject has been diagnosed as being at risk of developing CVD or cardiovascular thrombosis.

12. The method of claim 9, wherein the bodily sample is a plasma, blood, serum, urine, or other sample.

13. A method for characterizing a sample obtained from a subject having or at risk of having a cardiovascular thrombosis, comprising measuring a 12(S)-HETE and/or 13-HODE expression level in a sample (e.g., blood, plasma, urine, etc.) from the subject, and determining that the subject is experiencing cardiovascular thrombosis or is at risk of experiencing cardiovascular thrombosis if the measured 12(S)-HETE and/or 13-HODE expression level is higher compared to control 12(S)-HETE and/or 13-HODE expression levels from disease free or general population.

14. The method of claim 13, further comprising administering a treatment (e.g., a CVD therapeutic agent) to a subject characterized experiencing cardiovascular thrombosis or is at risk of experiencing cardiovascular thrombosis.

15. A method for detecting a cardiovascular thrombosis or risk of developing a cardiovascular thrombosis in a subject having or at risk of having a cardiovascular thrombosis, comprising measuring a 12(S)-HETE and/or 13-HODE expression level in a sample (e.g., blood, plasma, urine, etc.) from the subject, and determining that the subject is experiencing cardiovascular thrombosis or is at risk of experiencing cardiovascular thrombosis if the measured 12(S)-HETE and/or 13-HODE expression level is higher compared to control 12(S)-HETE and/or 13-HODE expression levels from disease free or general population. In some embodiments, the method further comprises administering a treatment (e.g., a CVD therapeutic agent) to a subject characterized experiencing cardiovascular thrombosis or is at risk of experiencing cardiovascular thrombosis.

16. The method of claim 15, further comprising administering a treatment (e.g., a CVD therapeutic agent) to a subject characterized experiencing cardiovascular thrombosis or is at risk of experiencing cardiovascular thrombosis.

17. A method for treating and/or preventing a cardiovascular thrombotic state in a subject (e.g., a human subject), comprising administering to a subject experiencing or at risk of experiencing a cardiovascular thrombotic state a therapeutic agent capable of decreasing 12(S)-HETE and/or 13-HODE expression levels and/or inhibiting 12-lipoxygenase and/or 15-lipoxygenase activity and/or inhibiting interaction between 12-lipoxygenase and/or 15-lipoxygeanse and arachidonic acid and/or linoleic acid.

18. The method of claim 17, wherein the therapeutic agent capable of decreasing 12(S)-HETE and/or 13-HODE expression levels and/or inhibiting 12-lipoxygenase activity and/or 15-lipoxygenase activity and inhibiting interaction between 12-lipoxygenase and arachidonic acid or 15-lipoxygenase and linoleic acid is selected from a small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

19. A method for hindering and/or inhibiting blood platelet activation in a subject, comprising administering to a subject experiencing or at risk of experiencing a cardiovascular thrombotic state a therapeutic agent capable of decreasing 12(S)-HETE and/or 13-HODE expression levels and/or inhibiting 12-lipoxygenase and/or 15-lipoxygenase activity and/or inhibiting interaction between 12-lipoxygenase and arachidonic acid or 15-lipoxygenase and linoleic acid.

20. The method of claim 19, wherein the therapeutic agent capable of decreasing 12(S)-HETE and/or 13-HODE activity levels and/or inhibiting 12-lipoxygenase activity or 15-lipoxygenase and/or inhibiting interaction between 12-lipoxygenase and arachidonic acid or 15-lipoxygenase and linoleic acid is selected from a small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).
